(19) United States
(12) Patent Application Publication
(10) Pub. No.: US 2006/0088591 A1
(43) Pub. Date: Apr. 27, 2006

(54) Tablets from a poorly compressible substance

(76) Inventors: Jinghua Yuan, Kingsport, TN (US); Nancy Meade Clipse, Mount Carmel, TN (US); Stephen Hong-Wei Wu, Chesterfield, MO (US)

Correspondence Address: Michael J. Blake;Eastman Chemical Company, P.O. Box 511, Kingsport, TN, 37662-5075, US

(21) Appl. No.: 10/972,095

(22) Filed: Oct. 22, 2004

(51) Int. Cl. A61K 9/20

(52) U.S. Cl. 424/464

(57) ABSTRACT

A solid composition suitable for forming into a tablet includes a pharmaceutically active substance in an amount sufficient to provide a therapeutic effect when administered; from about 0.2 to about 15 weight %, based on the total weight of the composition, of a water-soluble preparation of a fat-soluble vitamin; and from about 10 to 80 weight %, based on the total weight of the composition, of an excipient. Another aspect of the present invention is method for making the solid composition.

### FIELD OF THE INVENTION

The present invention relates to oral administration of medically active substances, and more particularly, to preparing poorly compressible medically active substances in the form of tablets.

### BACKGROUND OF THE INVENTION

A variety of substances, such as pharmaceutical active substances, intended for oral administration, are often formulated in a solid dosage form. As used herein the term “solid dosage form” is intended to refer to a presentation which is suitable in particular for oral or rectal administration and has any desired forms such as, for example, tablets, coated tablets, pastilles, pellets, granules and the like. The term “tablet” is well understood by not only those skilled in the art but is sufficiently familiar to the general public at large. Generally, the term “tablet” includes not only tablets per se but also similar discrete bodies, perhaps of other shapes and sometimes known by different names, such as “caplets” (e.g. capsule-shaped tablets), lozenges, and pills. As used herein, the term is also used to refer to mixtures of particulate solid materials, which have been brought together in various ways and finally compressed using one or more apparatus known to those skilled in the art so that the pharmaceutical active substances become compacted into shaped entities able to persist under normal handling conditions but disintegrate at the desired site, time or combination of both.

Typically, tablets will contain a medicament, an excipient such as a bulking agent, a binder to hold the tablet together, a disintegrant to promote the breaking-up of the tablet after ingesting and to release the medicine, and a lubricant to prevent the tablet from sticking to the tablet-punch. Additionally, the tablet may be coated with a bioactive or inert material to improve appearance, taste or improve shelf life of the tablet.

In a pharmaceutical context, the dose necessary at any one administration should desirably be given in the fewest possible tablets. However, consideration must also be given to the shape of the tablet and on the individual who is to swallow it. It is a relatively rare event that the dose of the pharmaceutical active substances necessary at any one administration should exceed the maximum swallowable size of tablet. A problem recently recognized by those skilled in the art is that as the amount of active matter to be given at any one administration so closely approaches the maximum swallowable size that the balance is not enough to accommodate tabletting aids (and possibly other ingredients) which are pharmacologically inert but whose presence is vital to the manufacture of a satisfactory tablet when, as so often, the compression characteristics of the active matter are poor.

Conventionally, tablets are defined in terms of their weight and not of their bulk. As to what precisely is the absolutely maximum “swallowable” size of tablet depends greatly on the shape of the tablet and on the individual who must swallow it. Generally, it is considered that the absolutely maximum “swallowable” size of tablet is about 1200 milligrams although lower sizes are preferred. Whatever the weight limit, any tablet that is to be swallowed must accommodate all its ingredients, not only the pharmaceutical active substances but also every other necessary or desirable type of ingredient, within that weight limit. Moreover, that consideration not only applies to tablets intended to be swallowed but to some extent also affects tablets of other kinds, because much of the available tablet manufacturing machinery is dimensioned to produce swallowable tablets.

In the manufacture of tablets, the final compression of each tablet takes place between the punches within a die. In making a tablet, generally a pregranulated mixture of particulate solid materials is loaded or placed in a die. The material is then compressed to a predetermined pressure and temperature which forms the tablet. When all the ingredients, including the active matter, have good compression characteristics, one may use dry granulation, the simplest and cheapest of techniques known, or a modified version thereof involving what is called preliminary slugging. However, if the compression characteristics of the mixture are poor, a defect attributable usually to the nature and/or amount of active matter present, one must resort on the technique known as wet granulation.

There is much art and skill in practicing the wet granulation technique, which generally involves no more than the incorporation of a granulating fluid into the mixed, powdery tablet ingredients, including at least some tabletting aids, in such an amount and manner as to convert them into a uniform, moist, coherent, non-pasty mass. This material is then formed into moist granules of fairly uniform size, usually by forcing the mass through a screen. Thereafter the moist granules are dried and rescreened to break down agglomerates, and finally blended with other tabletting aids to prepare the granulate ready for tabletting. In poorly compressible materials, where wet granulation is utilized, it is usually necessary to incorporate some appropriate amount of one or all of the conventional types of tabletting aids, such as binders, glidants, lubricants and disintegrants. One skilled in the art will understand the terms: “binder” to mean a substance which helps to bind the particles of powder together in a form suited to compaction and compression; “glidant” to mean a substance which aid filling of the particles and/or granules into the die before compression; “lubricant” to mean a substance which help the compressed tablets to leave the die; and “disintegrant” to mean a substance which help the tablet to disintegrate, and perhaps dissolve, when it reaches its ultimate destination, usually within the body.

One problem with wet granulation is that the “liquid” typically used to convert the poorly compressible substance into moist granules of fairly uniform size may interfere with the absorption of the pharmaceutically active substances or reduce its efficacy. For example, clorazepate dipotassium is used for the management of anxiety disorders and for short-term relief of symptoms of anxiety. A description of this drug is found, for example, in U.S. Pat. No. Re 28, 315. It has been found that minute amounts of water, i.e., about 0.3% w/v will hydrolyze the drug to nordiazepam. Additionally, in the case of such materials as water soluble vitamins, such as C and the B vitamins, water added to form a granular mix would reduce the shelf life of the material or reduce the efficacy of the material.

Another problem associated with poorly compressible substances is that unduly high pressures are needed to obtain a standard hardness of the tablet. However, the excessive pressures can lead to capping, i.e., top of the tablet breaking off, or lamination where layers of the tablet break apart. To overcome these problems, high levels of binder are required, which can inhibit disintegration of the tablet after administering.

Accordingly, there is a need for a composition and method for making a tablet from a poorly compressible material that will be acceptable for either aqueous or lipid soluble substances and that does not detrimentally affect the efficacy or shelf life of the active substance.

Surprisingly, it now has been found that by employing a modified version of conventional wet granulation there is provided a process for manufacturing tablets from poorly compressible pharmaceutical active substances which achieves this much desired goal without the addition of water to the poorly compressible material.

### SUMMARY OF THE INVENTION

The present invention is a solid composition suitable for forming a tablet. The solid composition comprises a poorly compressible, pharmaceutically active substance in an amount sufficient to provide a therapeutic effect when administered; from about 0.2 to about 10 weight % based on the total weight of the composition, of tocopherol polyethyleneglycol succinate; and from about 20 to 60 weight % based on the total weight of the composition, of an excipient, wherein the excipient is a substance other than tocopherol polyethyleneglycol succinate.

Another aspect of the present invention is a process for preparing a solid composition and particularly one suitable for tableting comprising the steps of melting a predetermined amount of tocopherol polyethyleneglycol succinate at a temperature of less than about 60° C.; mixing the melted tocopherol polyethyleneglycol succinate with a predetermined amount of the poorly compressible pharmaceutically active substance to form a substantially homogeneous particulate blend; cooling and screening the blend sufficiently to form a substantially uniform granulated material; and admixing an excipient to the substantially uniform granulated material.

It is an object of the present invention to provide a solid granulated composition that is suitable for the manufacture of tablets.

Another object of the present invention to provide a solid form of a poorly compressible pharmaceutical active substance that will be free flowing and compressible enough for pharmaceutical manufacturing processes such as tableting.

It is another object of the present invention to provide a method of making the solid granulated composition that is suitable for the manufacture of tablets.

### DETAILED DESCRIPTION OF THE INVENTION

In accordance with the present invention, the solid composition includes a solid, poorly compressible substance, and a poorly compressible pharmaceutically active substance in an amount sufficient to provide a therapeutic effect when administered. The term “poorly compressible” is well-known and understood by those skilled in the art, either from the general knowledge in the tableting field or by carrying out routine compression test on a standard tablet formulation including the ingredient. Poorly compressible materials will, for example, result in a tablet that caps, laminates or one that has greater than about 1 weight % loss after a friability test. The solid, poorly compressible substance can be hydrophilic, lipophilic, or amphiphilic. Non-limiting examples of pharmaceutical active substance include analgesics, anti-inflammatory agents, antihelmimthics, anti-arrhythmic agents, anti-bacterial agents, anti-viral agents, anti-coagulants, anti-depressants, anti-diabetics, anti-epileptics, anti-cancer agent, anti-fungal agents, anti-gout agents, anti-hypertensive agents, anti-malariale, anti-migrainc agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, antioxidant agent, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, a hormone, a steroid, steroid antagonist, a vitamin, essential fatty acids, non-essential fatty acids, and mixtures thereof.

The poorly compressible solid pharmaceutical active substances are known to those skilled in the art and can be determined by routine experimentation in the manufacture of a solid dosage form such as a tablet. As used herein the term “poorly compressible” would further include any solid granular or particulate matter that would have a weight loss of greater than about 1 weight % when tested for friability as described in the U.S. Pharmacopeias/National Formulary (USP 23/NF 18, pp 1981). Non-limiting examples of such poorly compressible material include but are not limited to: nalidixic acid, that is, 1-ethyl-1,4-dihydro-7-methyl-4-oxo-1,8-naphthyridine-3-carboxylic acid; paracetamol, that is, N-(4-hydroxyphenyl)acetamide, with or without methionine, that is 2-amino-4-(methylthio)butyric acid, hexopal, that is, myo-inositol hexa-3-pyridine-carboxylate; benorylate, that is, 2-(acetyloxy)benzoic acid 4-(acetylamino)phenyl ester; paracetamol methionate, that is, N-acetyl-para-aminophenyl N′-acetyl-methionate and ascorbic acid.

The amount of pharmaceutical active substance in the solid dosage form is desirably an amount sufficient to provide a therapeutic effect when administered. As discussed above, the size of a table typically is from about 250 to 1200 milligrams, with from about 400 to 850 milligrams being preferred. Generally, the amount of pharmaceutical active substances present in a tablet is from about 5 to 95 weight % of the tablet. Preferably, the amount of pharmaceutical active substances is from about 10 to 85 weight % of the tablet and, more preferably, it is from about 25 to 70 weight % of the tablet. In the case where the pharmaceutical active substance is susceptible to minute amounts of water, it is desirable that the tablet contain less than about 1 weight % water, and preferably the tablet contains less than about 0.05 to 0.1 weight %. As used herein, the term “weight %” is based on the total weight of the composition, unless specified otherwise.

The solid composition of the present invention further includes from about 0.2 to about 15 weight %, preferably from about 0.2 to about 10 weight %, more preferably from about 0.5 to about 8 weight %, and most preferably from about 0.5 to less than about 5 weight % of a water-soluble preparation of a fat-soluble vitamin. The water-soluble preparation of a fat-soluble vitamin suitable for use in the present invention are those disclosed in U.S. Pat. No. 3,102,078, and U.S. Pat. No. 2,680,749 the entire disclosures of which is incorporated herein by reference. Generally, U.S. Pat. No. 3,102,078 discloses a water-soluble preparation comprising up to about 3 parts by weight of a water-insoluble, fat-soluble vitamin composition mixed with 7 to 9 parts by weight of a vitamin E active, polyoxyethylene glycol ester of a tocopheryl ester of succinic acid. The polyoxyethylene glycol moiety has a molecular weight in the range of about 200 to 20,000, desirably of about 400 to 10,000, preferably of about 400 to 3000, and more preferably from about 400 to 1000. A water-soluble preparation of a fat-soluble vitamin is Vitamin E succinate polyethylene glycol 1000 is available from Eastman Chemical Company under the tradename Vitamin E TPGS™. Vitamin E TPGS™ is very stable and does not hydrolyze under normal conditions. Its therapeutic benefit has been well documented and is recognized by those skilled in the art.

In accordance with the present invention, the solid composition includes from about 10 to 80 weight %, preferably from 15 to 70 weight %, and more preferably from about 20 to 60 weight % of a pharmaceutically acceptable additive or excipient other than tocopherol polyethyleneglycol 1000 succinate. Such excipients may facilitate the production of the solid dosage form, such as a tablet, and/or modulate the properties of the final solid form. The excipient may be pre-coated or encapsulated. Examples of such excipients include, based on functionality, are as follows:

Anti-adherents (anti-sticking agents, glidants, flow promoters, lubricants) such as talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate (these anti-adherents should be present in amounts from 0.1-10 weight %, with a preferred range of 0.3-3.0 weight) %;

Anticoagulants, such as acetylated monoglycerides;

Antifoaming agents, such as long-chain alcohols and silicone derivatives;

Antioxidants, such as BHT, BHA, gallic acid, propyl gallate, ascorbic acid, ascorbyl palmitate, 4-hydroxymethyl-2,6-di-tert-butyl phenol, and tocopherol;

Binders (adhesives), i.e., agents that impart cohesive properties to powdered materials through particle-particle bonding, such as matrix binders (dry starch, dry sugars), film binders (PVP, starch paste, celluloses, bentonite, sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose);

Bufferants, where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid;

Chelating agents, such as EDTA and EDTA salts;

Coagulants, such as alginates;

Colorants or opaquants, such as titanium dioxide, food dyes, lakes, natural vegetable colorants, iron oxides, silicates, sulfates, magnesium hydroxide and aluminum hydroxide;

Cryoprotectants, such as trehelose, phosphates, citric acid, tartaric acid, gelatin, dextran and mannitol;

Diluents or fillers, such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose;

Disintegrants or super disintegrants, such as cross-linked sodium carboxymethyl cellulose (Ac-Di-Sol), sodium starch glycolate (Explotab, Primojel), and cross-linked polyvinylpolypyrrolidone (Plasdone-XL), clays, gums, cellulose, cellulose derivatives, alginates, sodium starch glycolate and microcrystalline cellulose. These materials should be present in the range of 3-15% (w/w), with a preferred range of 5-10% (w/w);

Flavorants or desensitizers, such as spray-dried flavors, essential oils and ethyl vanillin;

Plasticizers, such as polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate;

Preservatives, such as ascorbic acid, boric acid, sorbic acid, benzoic acid, and salts thereof, parabens, phenols, benzyl alcohol, and quaternary ammonium compounds;

Sweeteners, including natural sweeteners such as maltose, sucrose, glucose, sorbitol, glycerin and dextrins, and artificial sweeteners, such as aspartame, saccharine and saccharine salts; and

Materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum arabic); spermaceti; natural or synthetic waxes; carnuaba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based shellacs (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate trimellitate, carboxymethylethyl cellulose, hydroxypropylmethyl cellulose phthalate); inorganics, such as dicalcium phosphate, hydroxyapitite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. The amounts of such additives can be readily determined by one skilled in the art, according to the particular properties desired.

The solid dosage form may be coated with one or more enteric coatings, seal coatings, film coatings, barrier coatings, compress coatings, fast disintegrating coatings, extended release coating, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be affected through favorable diffusion, dissolution, erosion, ion-exchange, osmosis or combinations thereof.

As used herein, the term “extended release coating” as used herein means a coating designed to affect delivery over an extended period of time. Preferably, the extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

The methodology utilized in coating the solid dosage form is not critical and is generally known to those skilled in the art. For example, the tablet may be coated using spray coating, fluidized bed coating, and pan coating. Moreover, It should be appreciated that any of the components of the compositions of the present invention can be used as supplied commercially.

In preparing the solid composition of the present invention, the following method is employed: a) melting a predetermined amount of the water-soluble preparation of a fat-soluble vitamin, such as tocopherol polyethyleneglycol succinate, preferably at a temperature of less than about 60° C.; b) contacting the melted tocopherol polyethyleneglycol succinate with a predetermined amount of a pharmaceutically active substance to form a substantially homogeneous particulate blend; c) cooling and screening the particulate blend sufficiently to form a substantially uniform granulated material; and d) admixing an excipient to the substantially uniform granulated material. As used herein, the term “substantial” or “substantially” means that greater than about 80%, preferably greater than about 90% and more preferably greater than about 95% of the material is of a uniform size and/or uniform concentration. These post-granulation excipient(s) will usually consist of or include one or more components or mixture of components capable of imparting an effervescent character to the final tablet, but in that event all the parameters of the process must of course be chosen so as to form the final tablet without detriment to its desired character or effect. The process of the present invention also extends, of course, to tablettizing the granulates and also tablets made therefrom whenever prepared in or by the process described herein.

One skilled in the art will readily recognize that the granulation method of this invention naturally can be employed in making a tablet of any kind of active matter, even easily compressible pharmaceutical active substances. However, this granulation method of the present invention has been developed with the particular objective of improving formation of tablets of poorly compressible active matter.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis of the total composition unless stated otherwise.

In the examples that follow the ingredients consisted of:
a) 30-70 weight % ascorbic acid (available from Weisheng Pharma, Shijiazhuang, China) was chosen as the model active for the tablets due to its poor compressible characteristics;
b) 2-10 weight % Vitamin E 1000 NF TPGS (available from Eastman Chemical Company, Kingsport, Tenn.), was used as the water-soluble preparation of a fat-soluble vitamin which functionally acted as a thermal binder/lubricant;
c) 20-68 weight % microcrystalline cellulose (MCC, Avicel PH 101, FMC, Newark, Del.) was used functionally as a binder/filler; and
d) about 4 weight % fume silica, based on the weight of the MCC, (SYLOID, available from W. R. Grace, Columbia, Md.) was used functionally as flowing aid.

Two granulation formulation methods were conducted. The procedures for Method 1 were:
1. Weight the ascorbic acid, TPGS, MCC as needed.
2. Heat the active to 60° C. and maintain the temperature in an oven.
3. Heat the TPGS to 60° C. and maintain at 60° C. in a mixing bowl.
4. Blend the warmed active into the melted TPGS using a mixer with a wire stirrer (N50, Hobart Manufacturing, London England) for five minutes at 60° C.
5. Transfer the blended material to an unheated mixing bowl, blend for eight more minutes, and let the blend to cool to room temperature.
6. After cooling to room temperature, sieve the material through 14 mesh sieve to remove any lumps.
7. Add the MCC to the sived material and blend for three minutes in the unheated mixing bowl.
8. Add the fume silica and blend for three minutes in an unheated mixing bowl.
   Preparing Tablets From the Material Prepared Using Method 1:

Tablets of approximately 600 milligrams (mg) were made using a 16-station rotary tablet press (D3B, available from Manesty, England). A sample size of 40 tablets was used to determine friability. If the tablets in the sample passed the friability test, more tablets were prepared under this compression force, otherwise the compression force was adjusted to make tablet hard enough to pass friability test. The compression force and ejection force used to make tablets was recorded. After tableting, examine the dies and punches to observe any adherent materials.

The procedures for Method 2 were:
1. Weight ascorbic acid, MCC, TPGS, and fume silica as needed.
2. Heat the active and MCC to 60° C. maintain at this temperature in an oven.
3. Heat the TPGS to 60° C. and maintain at 60° C. in a heated mixing bowl.
4. Mix the warmed active and MCC for three minutes in an unheated mixing bowl using wire stirrer.
5. Transfer the material from 4 to the melted TPGS in the heated mixing bowl and stir for five minutes at 60° C. to form a substantially uniform blend.
6. Transfer the blend from 5 to a unheated mixing bowl, and stir for eight minutes at room temperature. Allow the blend to cool to room temperature.
7. After cooling to room temperature, sieve the material through 14 mesh sieve to remove any lumps.
8. Transfer the material from 7 to the unheated mixing bowl, add the fume silica, and blend for three minutes at room temperature.

The procedure for preparing tablets from the material prepared using Method 2 was the same as described above for Method 1.

The powder flowability, compression and ejection force were used to evaluate the feasibility of the processes.

Tablet weight, friability, hardness and thickness of the tablets were evaluated as measures for tablet quality.

USP dissolution test method apparatus II was employed to determine ascorbic acid release profiles in pH 1.2 buffer. The pH 1.2 buffer solution was prepared according to the methods described in USP 25/NF 20. The dissolution tests were performed in 1000 ml of the dissolution medium that maintained at 37° C. The paddle's stirring rate was set at 50 rpm. The ascorbic acid tablets with different TPGS contents (2, 5, and 10 weight %) were tested. Six tablets were randomly chosen for the testing, the average was reported to represent the release profile. The results appear in Table I below.

TABLE 1
Percentage of Ascorbic Acid Released

| time (minute) | 2% TPGS | 5% TPGS | 10% TPGS |
|---|---|---|---|
| 0 | 0 | 0 | 0 |
| 2.9 | 65.51 | 9.92 | 11.92 |
| 7.5 | 89.50 | 28.24 | 29.75 |
| 10 | 92.45 | 37.59 | 37.29 |
| 15 | 95.80 | 55.87 | 49.80 |
| 20 | 97.35 | 72.92 | 59.93 |
| 30 | 98.74 | 92.70 | 74.71 |
| 45 | 99.52 | 99.65 | 89.06 |
| 60 | 99.86 | 99.95 | 97.29 |
| 80 | 99.97 | 99.97 | 99.69 |
| 100 | 100 | 100 | 99.98 |
| 120 | 100 | 100 | 100 |

### EXAMPLE 1

In accordance with Method 1, a granulated material was prepared having ascorbic acid (840.03 g), TPGS (120.00 g), MCC (230.60 g), and fume silica (9.63 g). The mixture was free flowing from the hopper to the tabletting machine. To make 600 mg tablets, it required 3900 lbs of compression force, and the ejection force was 20 lb. Twenty tablets were randomly chosen for friability testing (Vanderkamp, Model 10809). Friability is calculated as follows:
friability (%)=(weight of 20 tablets before testing−weight of 20 tablets after testing)/(weight of 20 tablets before testing)×100%.

The results in Table 2 below.

TABLE 2

| | 20 tablets | 20 tablets |
|---|---|---|
| before testing | 11.91 g | 12.00 g |
| after testing | 11.91 g | 11.99 g |
| friability | 0% | 0% |

One hundred tablets were randomly chosen to test hardness and thickness of the tablets, The average hardness and thickness of the tablets were determined to be 84 Newtons (N) and 2.34 mm, respectively. The tablets became harder with time, as 84 N for fresh tablets, 98 N for tablets after one hour, 106 N for tablet after one day.

### EXAMPLE 2

In accordance with Method 2, a granulated material was prepared having ascorbic acid (300.23 g), TPGS (60.12 g), MCC (230.43 g), and fume silica (9.62 g). The mixture was free flowing from the hopper to the tabletting machine. To make 600 mg tablets, it required 3400 lbs of compression force, and the ejection force was 70 lb. The friability results are listed in Table 3 below.

TABLE 3

| | 20 tablets | 20 tablets |
|---|---|---|
| before testing | 12.10 g | 12.02 |
| after testing | 12.10 g | 12.02 |
| friability | 0% | 0% |

The average hardness and thickness of 20 tablets were determined as 106 N and 2.50 mm. The tablets became harder with time, 106 N for fresh sample, 117 N after one hour, and 117 N after six hours.

### EXAMPLE 3

The ingredients for making tablets were prepared according to the procedures of Method 1. The points at which no flowability and process problems were observed during tablet making was called the working point. The working points are listed in Table 4 below.

### EXAMPLE 4

The ingredients for making tablets were prepared according to the procedures of Method 2. The points at which no flowability and process problems were observed during tablet making was called the working point. The working points are listed in Table 5 below.

TABLE 4

| | | | | | Tablet | | | |
|---|---|---|---|---|---|---|---|---|
| Vitamin | TPGS | MCC | Compression | Ejection | Weight | Friability | Hardness | Thickness |
| C (%) | (%) | (%) | (lb) | (lb) | (mg) | (%) | (N) | (mm) |
| 0.7 | 0.05 | 0.25 | 4200 | 80 | 612 | 0 | 133 | 3.35 |
| 0.5 | 0.05 | 0.45 | 3300 | 80 | 602 | 0 | 256 | 3.34 |
| 0.5 | 0.05 | 0.45 | 1900 | 60 | 613 | 0 | 318 | 3.42 |
| 0.7 | 0.1 | 0.2 | 3900 | 20 | 602 | 0 | 84 | 3.34 |
| 0.5 | 0.05 | 0.45 | 1900 | 60 | 611 | 0 | 263 | 3.23 |
| 0.7 | 0.02 | 0.28 | 2600 | 180 | 611 | 0 | 122 | 3.18 |
| 0.5 | 0.02 | 0.48 | 1900 | 210 | 602 | 0.12 | 298 | 3.19 |
| 0.3 | 0.02 | 0.68 | 600 | 220 | 602 | 0.08 | 380 | 3.96 |

TABLE 5

| | | | | | Tablet | | | |
|---|---|---|---|---|---|---|---|---|
| Vitamin | TPGS | MCC | Compression | Ejection | Weight | Friability | Hardness | Thickness |
| C (%) | (%) | (%) | (lb) | (lb) | (mg) | (%) | (N) | (mm) |
| 0.5 | 0.05 | 0.45 | 2900 | 160 | 606 | 0 | 186 | 3.56 |
| 0.5 | 0.05 | 0.45 | 3100 | 160 | 617 | 0 | 183 | 3.61 |
| 0.3 | 0.1 | 0.6 | 3200 | 90 | 603 | 0 | 182 | 2.61 |
| 0.3 | 0.05 | 0.65 | 1200 | 140 | 599 | 0 | 209 | 2.59 |
| 0.5 | 0.05 | 0.45 | 3200 | 160 | 596 | 0 | 165 | 2.44 |
| 0.5 | 0.1 | 0.4 | 3400 | 70 | 601 | 0 | 106 | 2.50 |
| 0.5 | 0.02 | 0.48 | 3400 | 200 | 606 | 0 | 247 | 3.47 |
| 0.52 | 0.1 | 0.38 | 3300 | 50 | 609 | 0 | 58 | 3.7 |
| 0.52 | 0.05 | 0.43 | 3600 | 160 | 611 | 0 | 87 | 3.6 |
| 0.52 | 0.02 | 0.46 | 3400 | 240 | 600 | 0 | 127 | 3.5 |

Having described the invention in detail, those skilled in the art will appreciate that modifications may be made to the various aspects of the invention without departing from the scope and spirit of the invention, and equivalents thereof, disclosed and described herein. Moreover, all patents, patent applications, publications, and literature references presented herein are incorporated by reference in their entirety for any disclosure pertinent to the practice of this invention.

We claim:

1. A solid composition comprising:
a. a pharmaceutically active substance in an amount sufficient to provide a therapeutic effect when administered;
b. from about 0.2 to about 15 weight %, based on the total weight of the composition, of a water-soluble preparation of a fat-soluble vitamin; and
c. from about 10 to 80 weight %, based on the total weight of the composition, of an excipient, wherein said excipient is a substance other than tocopherol polyethyleneglycol 1000 succinate.

2. The solid composition of claim 1 wherein said pharmaceutically active substance is a poorly compressible substance selected from the group consisting of analgesics, anti-inflammatory agents, antihelmimthics, anti-arrhythmic agents, anti-bacterial agents, anti-viral agents, anti-coagulants, anti-depressants, anti-diabetics, anti-epileptics, anti-cancer agent, anti-fungal agents, anti-gout agents, anti-hypertensive agents, anti-malariale, anti-migrainc agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, antioxidant agent, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, a hormone, a steroid, steroid antagonist, a vitamin, essential fatty acids, non-essential fatty acids, and mixtures thereof.

3. The solid composition of claim 1 wherein the water-soluble preparation of a fat-soluble vitamin is Vitamin E polyethylene glycol succinate wherein the polyoxyethylene glycol moiety has a molecular weight in the range of about 200 to about 20,000.

4. The solid composition of claim 1 wherein the water-soluble preparation of a fat-soluble vitamin is Vitamin E polyethylene glycol succinate wherein the polyoxyethylene glycol moiety has a molecular weight in the range of about 400 to about 3000.

5. The solid composition of claim 1 wherein the water-soluble preparation of a fat-soluble vitamin is Vitamin E polyethylene glycol succinate wherein the polyoxyethylene glycol moiety has a molecular weight in the range of about 400 to about 2000.

6. The solid composition of claim 3 wherein the polyoxyethylene glycol moiety has a molecular weight of about 1000 and the amount of tocopherol polyethyleneglycol 1000 succinate is from about 0.2 weight % to about 10 weight %.

7. The solid composition of claim 6 wherein the amount of tocopherol polyethyleneglycol 1000 succinate is from about 0.5 weight % to about 8 weight %.

8. The solid composition of claim 6 wherein the amount of tocopherol polyethyleneglycol 1000 succinate is from about 0.5 weight % to about 5 weight %.

9. The solid composition of claim 1 wherein said excipient is selected from the group consisting of an anti-sticking agent, a glidant, a flow promoter, a lubricant, an anticoagulant, an antifoaming agent, an antioxidant, a binder, a bufferant, a chelating agent, a coagulant, a colorant, a cryoprotectant, a diluent, a filler, a disintegrant, a flavorant or sweetner, a plasticizer, a preservative and mixtures thereof.

10. The solid composition of claim 9 wherein the amount of said excipient is from about 15 to 70 weight %, based on the total weight of the solid composition.

11. The solid composition of claim 9 wherein the amount of said excipient is from about 20 to 60 weight %, based on the total weight of the solid composition.

12. A method for preparing a solid composition of claim 1 comprising the steps of:
a. melting a predetermined amount of tocopherol polyethyleneglycol succinate at a temperature of less than about 60° C.;
b. contacting said melted tocopherol polyethyleneglycol succinate with a predetermined amount of a pharmaceutically active substance to form a substantially homogeneous particulate blend;
c. cooling and screening said particulate blend sufficiently to form a substantially uniform granulated material; and
d. admixing an excipient to said substantially uniform granulated material.

13. The method of claim 12 further comprising:
e. compressing said substantially uniform granulated material in an apparatus adapted for forming a tablet to form a tablet.

14. The process of claim 12 further comprising admixing an additive with said pharmaceutically active substance prior to contacting said melted tocopherol polyethyleneglycol succinate.

15. The process of claim 14 wherein said additive is selected from the group consisting of a lubricant, anti-sticking agent, a lubricant, an anticoagulant, an antifoaming agent, an antioxidant, a binder, a bufferant, a chelating agent, a coagulant, a colorant, a cryoprotectant, a diluent, a filler, a disintegrant, a flavorant, a sweetener, a plasticizer, a preservative and mixtures thereof.

16. A solid pharmaceutical of claim 1 prepared by the process of claim 10.

17. A solid pharmaceutical of claim 1 prepared by the process of claim 12.

18. The solid pharmaceutical of claim 16 or 17 wherein the amount of tocopherol polyethyleneglycol succinate is from about 0.2 weight % to about 10 weight %.

19. The solid pharmaceutical of claim 18 wherein the amount of tocopherol polyethyleneglycol succinate is from about 0.5 weight % to about 5 weight %.

20. The solid pharmaceutical of claim 16 or 17 wherein said excipient is selected from the group consisting of an anti-sticking agent, a glidant, a flow promoter, a lubricant, an anticoagulant, an antifoaming agent, an antioxidant, a binder, a bufferant, a chelating agent, a coagulant, a colorant, a cryoprotectant, a diluent, a filler, a disintegrant, a flavorant or sweetner, a plasticizer, a preservative and mixtures thereof.

21. The solid pharmaceutical of claim 18 wherein the amount of said excipient is from about 15 to 70 weight %, based on the total weight of the solid composition.
